(19) United States
(12) Patent Application Publication
(10) Pub. No.: US 2023/0166102 A1
(43) Pub. Date: Jun. 1, 2023

(54) SHAPE-MEMORY ALLOY AND POLYMER ELECTRODE ARRAY FOR MINIMALLY-INVASIVE SPINAL CORD AND BRAIN STIMULATION AND RECORDING

(71) Applicants: Brown University, Providence, RI (US); Rhode Island Hospital, Providence, RI (US)

(72) Inventors: Vikas SRIVASTAVA, East Greenwich, RI (US); Albert TELFEIAN, Providence, RI (US); Shailen SAMPATH, Lincoln, RI (US)

(21) Appl. No.: 17/997,329

(22) PCT Filed: Apr. 28, 2021
(86) PCT No.: PCT/US2021/029704

§ 371 Date: Oct. 27, 2022

Related U.S. Application Data
(60) Provisional application No. 63/106,062, filed on Oct. 27, 2020.
(60) Provisional application No. 63/016,716, filed on Apr. 28, 2020.

(51) Int. Cl. A61N 1/05; A61N 1/36

(52) U.S. Cl. CPC A61N 1/0529; A61N 1/0551; A61N 1/36062

(57) ABSTRACT

An electrode array includes a removable outer sheath, an insulation layer, the outer sheath surrounding the insulation layer, and a central lumen surrounded by the insulation layer, the central lumen including a metal alloy of nickel and titanium.

### CROSS REFERENCE TO RELATED APPLICATIONS

This application claims benefit from U.S. Provisional Patent Application Ser. No. 63/016716, filed Apr. 28, 2020, and U.S. Provisional Patent Application Ser. No. 63/106062, filed Oct. 27, 2020, each of which is incorporated by reference in its entirety.

### STATEMENT REGARDING GOVERNMENT INTEREST

None.

### BACKGROUND OF THE INVENTION

The present invention relates generally to electrode arrays, and more particularly to a shape-memory alloy and polymer electrode array for minimally-invasive spinal cord and brain stimulation and recording.

In general, it is advantageous to have an electrode system for spine and brain stimulation and recording that can be inserted using a minimally invasive procedure and that can achieve high coverage of intended neuronal targets internally after deployment. However, such electrode systems have been slow to develop, partially due to problems with the materials used in existing metallic and ceramic probes. As a result, most methods to stimulate and record neuronal activity cause unnecessary tissue damage.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In an aspect, the invention features an electrode array including a removable outer sheath, an insulation layer, the outer sheath surrounding the insulation layer, and a central lumen surrounded by the insulation layer, the central lumen including a metal alloy of nickel and titanium.

In another aspect, the invention features an electrode array including an electrode array including a removable outer sheath, an insulation layer, the removable outer sheath surrounding the insulation layer, and a central lumen surrounded by the insulation layer, the central lumen including thermoset shape-memory polymer.

In still another aspect, the invention features a method including adapting biocompatible shape-memory alloy-based wires to an electrode array, inserting the adapted electrode array in a minimally invasive fashion through a needle into a part of a human body, and transforming the adapted electrode array electrode shape using heat to maximize a coverage area of the part of the human body.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 illustrates a cross-section of an exemplary modified cylindrical lead.

FIG. 2 illustrates an exemplary view of the three steps in inserting a modified cylindrical lead for dorsal column stimulation into the epidural space that can allow for minimally invasive insertion of an electrode with more coverage.

FIG. 3 illustrates three exemplary examples of how the heat induced shape memory ability of our modified electrode could be activated.

FIG. 4 illustrates the exemplary thermally induced shape memory ability of shape memory polymers.

FIG. 5 illustrates the exemplary shape memory ability of Nitinol as it is deformed into a temporary shape and heated to allow for recovery to its original geometry in four steps.

FIG. 6 illustrates two exemplary example geometries that a nitinol component of a cylindrical lead can take.

### DETAILED DESCRIPTION

The subject innovation is now described with reference to the drawings, wherein reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Neuromodulation is a rapidly expanding field in clinical neuroscience. Today, electrode arrays in the form of leads, grids, and paddles are placed to stimulate and record in the brain and spinal cord for the treatment of neurologic and psychiatric diseases that include, for example, epilepsy, Parkinson's, obsessive-compulsive disorder and pain. In order to maximize electrode coverage in the brain and the spine, typically, a craniotomy or laminectomy must be performed in order to place a large grid or paddle electrode to cover the brain or spine.

The present invention is an adaption to traditional electrode arrays: biocompatible shape-memory alloy and/or thermoset and thermoplastic polymer based wires, sheathings, hinges or other compact geometries are used to insert an electrode array in a minimally invasive fashion through a needle or a small window in the spine or skull and then using the shape memory response of the shape memory alloy and/or polymer to transform that electrode shape into a shape that covers a more desirable area of the brain or spinal cord. In our design, the use of elasticity of metal wires can provide higher level of shape changing force where needed. The shape changes of biocompatible shape memory alloys and polymers are mainly activated through thermal response. When the components reach above a certain transition temperature range a shape change is induced.

By way of one example, shape memory alloys can be used to optimize dorsal column stimulation. Dorsal column stimulation for chronic pain is a neuromodulation procedure whereby an electrode array can be placed on the dorsal columns of the spinal cord either percutaneously by sliding small straight electrodes through a needle and advancing them epidurally using X-ray over the appropriate spinal level, or through an open procedure where an electrode paddle is placed through an open laminectomy over the appropriate level. The present invention bridges a gap between the open and percutaneous procedure. The shape-memory alloy or polymer-based electrode system of the present invention can be placed percutaneously but have the same superior coverage of the spine level for stimulator that an open paddle electrode does. In the case of the shape memory alloy Nitinol, Nitinol is set to a desired shape ex vivo and deformed into a linear shape before insertion. Nitinol is able to retain its linear deformed shape outside the environment of the body. It's special characteristics as a thermally induced shape memory alloy allow it to maintain its shape of deformation when it is underneath its transition temperature. After the shape memory alloy Nitinol is placed in an environment above its transition temperature the material will exhibit superelastic characteristics and revert to its desired initially set shape. Therefore, once the Nitinol lead is inserted into the body it will revert to its initial programmed shape meant to optimize the leads coverage for dorsal column stimulation. The phase change that occurs below Nitinol's shape transition temperature and also once Nitinol is heated above it's transition temperature. The present invention can use body temperature as a natural way to heat the shape memory alloy and polymer above it's shape transitioning temperature without a need for external heat source.

In one embodiment, the present invention uses the shape memory effects of Nitinol, a metal alloy of nickel and titanium, or other shape memory alloy or shape memory polymer, to create a modified cylindrical lead that will expand to more closely resemble area coverage of a paddle lead or geometry that best targets specific dermatomes dictated by industrial preference, once it is exposed to the body's environment.

The modified cylindrical lead can be inserted in a minimally invasive fashion utilizing a percutaneous technique similar to cylindrical lead placement, before expanding in situ. The modified cylindrical lead provides more coverage than current cylindrical leads, reduces the need for costly and invasive laminectomies, and increases accessibility to the procedure to not only spine surgeons but all pain management physicians.

The modified cylindrical lead also can be used for applications beyond dorsal column targeted spinal cord stimulation, including stimulation of a peripheral nerve, stimulation of the dorsal root, and recording of nerves. In one implementation, this is achieved by utilizing the shape memory alloy Nitinol (NiTi).

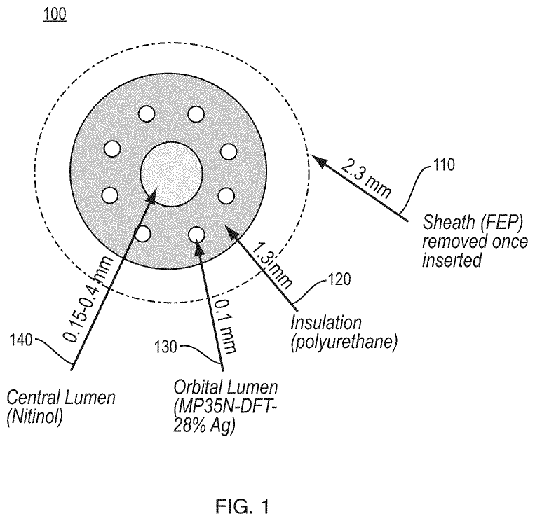

In FIG. 1, a cross-section of an exemplary modified cylindrical lead 100 for dorsal column stimulation is illustrated and includes an outer sheath 110 that is removed when the modified cylindrical lead 100 is inserted. Within the outer sheath 110 is an insulation layer 120 that includes a number of orbital lumen 130 that carry conductive components that deliver the stimulation. The insulation layer 120 surrounds a central lumen 140 that in one implementation uses the shape memory alloy Nitinol. In another implementation the central lumen 140 can have other shape memory alloys or polymers.

As described above, Nitinol (NiTi), is a metal alloy used in many medical applications due to its biocompatibility and super-elasticity. The present invention uses shape memory effect where the material undergoes a ‘phase change’ above a certain temperature that transitions the material from a material phase with a lower stiffness into a stiffer super-elastic material that takes on the original shape that it was processed in. Below body temperature, the modified cylindrical lead 100 can be manufactured in a shape that resembles a paddle lead spinal coverage, or any shape that would most optimally target a desired region. In the geometries produced, the modified cylindrical lead 100 is straightened, placed into an introducer sheath, and inserted into the epidural space percutaneously similar to a cylindrical electrode. Once inserted to the desired location, the sheath is removed and the Nitinol upon reaching body temperature reverts the lead back into a shape that provides optimal coverage.

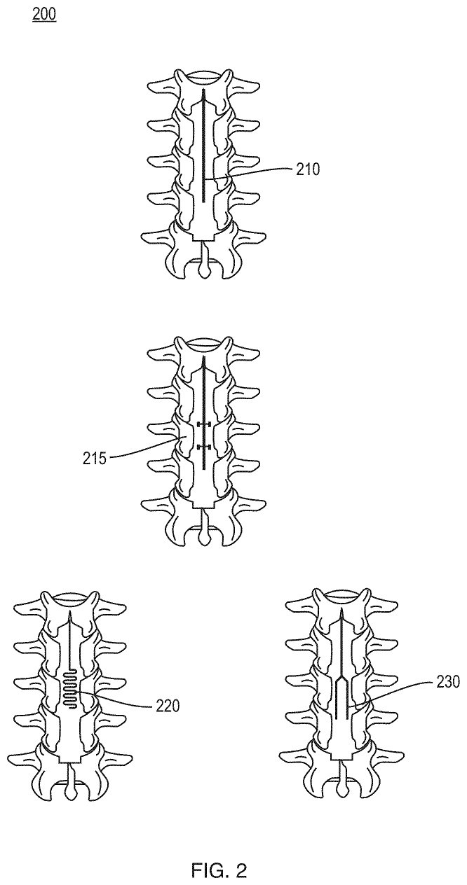

In FIG. 2, an exemplary representation of insertion of the modified cylindrical lead for dorsal column stimulation is illustrated. At 210, a strait modified cylindrical lead is inserted percutaneously. At 215, the lead is navigated up the epidural space to its desired region. At 220, the introducer sheath is removed and the modified electrode is allowed to revert the shape 230 it was processed in. At 220, the shape is in a s-shaped zig zag geometry. For demonstration purposes, here we illustrate two cylindrical leads 230, but more than two leads can be used in our application.

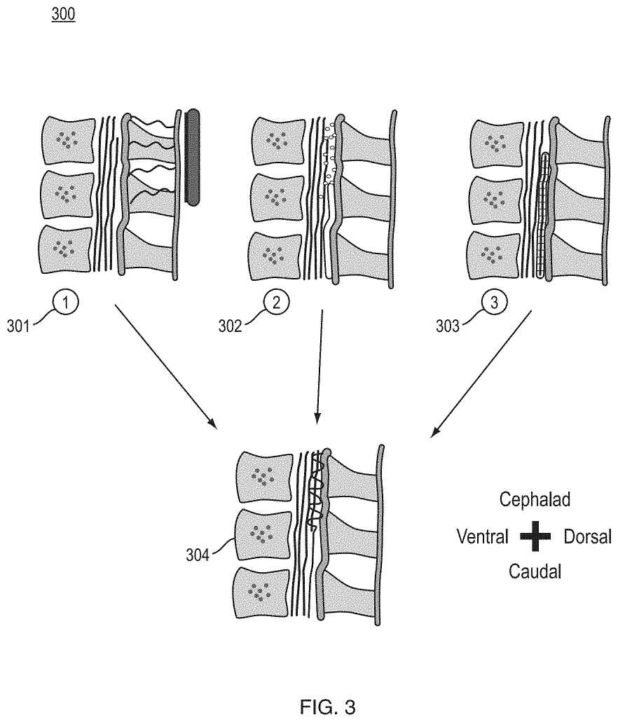

In FIG. 3, an exemplary representation 300 of how shape change of a modified cylindrical electrode for dorsal column stimulation in the body can be induced is illustrated. The recovery of the shape memory alloy or polymer is not restricted to heating the components to body temperature. The shape memory alloy and polymer can be synthesized and processed in a way which allows for customizability in the temperature range which induces a phase change.

In FIG. 3, an external heat source 301 can be used to induce shape change of the modified electrode in certain designs. In 302, cold saline water is administered along with the electrode which prevents recovery. In yet another example 303, a sheath can be used to prevent premature recovery of the modified electrode. These are exemplary methods that can be used to allow control over when shape recovery occurs 304.

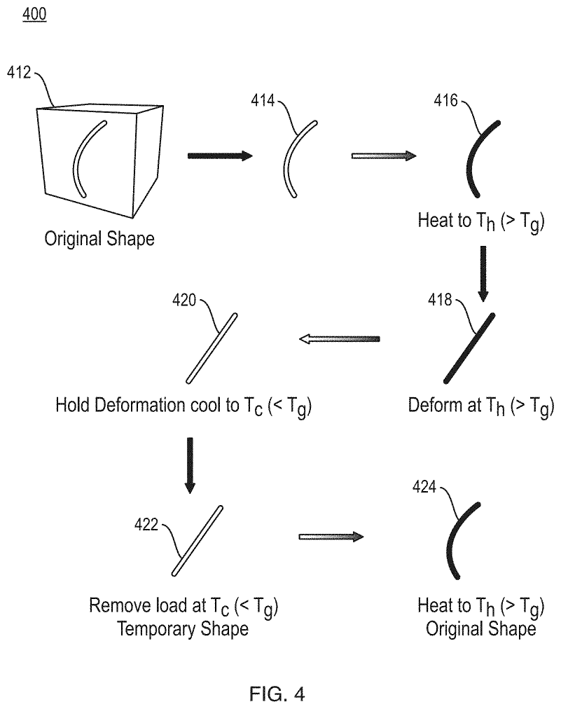

In FIG. 4, an exemplary representation of shape memory hinge fabrication 400 is shown. A thermoset shape-memory polymer 412 in its original shape 414 is heated to a high temperature Tₕ (>T_{g}) to provide a heated shape 416, where T_{g} is its glass transition temperature. The heated shape 416 is deformed to a deformed (straight) shape 418 at Tₕ (>T_{g}). The deformed shape 418 is cooled to a lower temperature T_{c} (>T_{g}) to hold a shape 420. Removing the load at T_{c} (>T_{g}) preserves the temporary shape 422. The shape 422 can be inserted through minimally invasive procedures and a body temperature or external stimulus can change the shape of hinge. Heating the temporary shape 422 at Tₕ (>T_{g}) returns the temporary shape 422 to its original thermoset shape-memory polymer shape 424.

In summary, when the original shape is brought to a temperature above the glass transition temperature, it is able to deform in its rubbery soft state. The deformation can then be held when the material is cooled. The polymer returns to its original shape when heated above glass transition temperature. The same process applies for a shape-memory alloy changing shape to a desired shape.

In one specific example, a shape memory polymer is made using Tert-butyl acrylate (tBA), poly(ethylene glycol)n dimethacrylate (PEGDMA), and photoinitiater 2,2-dimethoxy-2-phenylacetophenone. The constituents are 89.2%, 10%, and 0.2% by weight, respectively. Shape memory polymer sheets are made via mold of a petri dish and top made of glass slides, in which the mixed and autoclaved (for 10 minutes) solution is placed in between the two glass surfaces. This mold is separated by 1-2 spacers also made of glass slides, each 0.15 inches (3.175 mm) in thickness. Afterwards the solution is UV cured for 15 minutes and oven cured for 60 minutes at 90° C. to get the final shape memory polymer in sheet form. The semi-circular shaped hinges with rectangular cross section are then cut from this sheet.

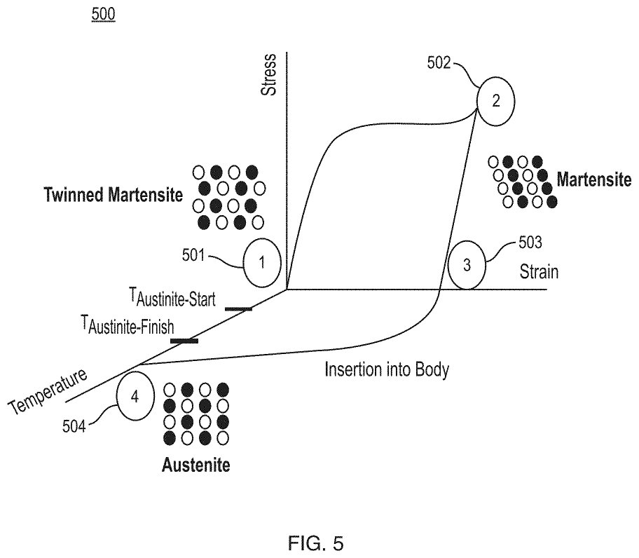

In FIG. 5, an exemplary illustration 500 of the phase change of Nitinol is illustrated. At 501, the Nitinol is in a twinned martensite shape at temperatures below a specific transition temperature range. Below the transition range in a twinned martensite phase, Nitinol can be easily deformed at 502 up to 11% strain into another geometry, such as a linear shape for easier percutaneous insertion of an electrode. This will cause Nitinol to go from a twinned martensite to a martensite phase, which will maintain its ‘deformed’ shape at 503 until heated above its transition range. Once Nitinol is heated about its transition temperature it will convert to an austinite phase at 504 and will take the shape it was in when it was in its twinned martensite phase.

To obtain the original austinite shape of the Nitinol, the Nitinol can either be obtained in the shape that is desired, machined in the desired shape from a blanket form, or commercially available Nitinol can be set to our desired shape. To set the austinite shape of the Nitinol, the material must be heated to 500° C. for one to three hours fixed in the shape for intended use.

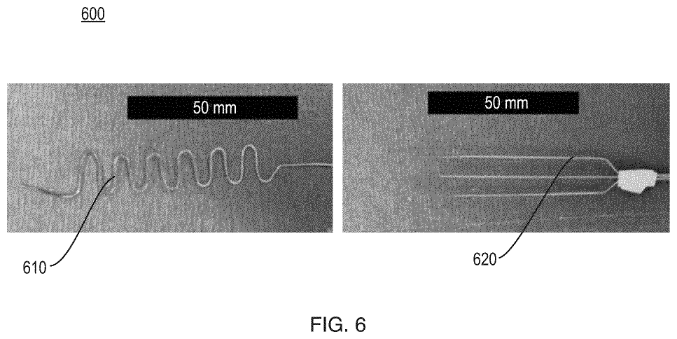

In FIG. 6, exemplary images of example geometries of a nitinol component of the cylindrical lead are illustrated. Image 610 displays a potential s-shaped geometry. Image 620 displays a trident shaped geometry. This geometry could have as few as two and as many parallel cylindrical leads as wanted that would break apart from each other when exposed to the bodies environment and allowed to recover. These geometries are examples and are not exhaustive of all the potential geometries the shape memory alloy and polymer based cylindrical leads could take.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. An electrode array comprising:
a removable outer sheath;
an insulation layer, the outer sheath surrounding the insulation layer; and
a central lumen surrounded by the insulation layer, the central lumen including a metal alloy of nickel and titanium.

2. The alloy electrode array of claim 1 wherein the outer sheath is removable.

3. The electrode array of claim 2 the insulation layer includes a number of orbital lumen.

4. The electrode array of claim 3 wherein the metal alloy of nickel and titanium is replaced with other shape memory alloys.

5. An electrode array comprising:
an electrode array including a removable outer sheath, an insulation layer, the removable outer sheath surrounding the insulation layer; and
a central lumen surrounded by the insulation layer, the central lumen including thermoset shape-memory polymer.

6. The electrode array of claim 5 the insulation layer includes a number of orbital lumen.

7. The electrode array of claim 6 wherein the outer sheath comprises fluorinated ethylene propylene (FEP).

8. The electrode array of claim 7 wherein the insulation layer comprises polyurethane.

9. A method comprising:
adapting biocompatible shape-memory alloy-based wires to an electrode array;
inserting the adapted electrode array in a minimally invasive fashion through a needle into a part of a human body; and
transforming the adapted electrode array electrode shape using heat to maximize a coverage area of the part of the human body.

10. The method of claim 9 wherein the biocompatible shape-memory alloy comprises a metal alloy of nickel and titanium.

11. The method of claim 10 wherein the metal alloy of nickel and titanium is Nitinol.

12. The method of claim 11 wherein the heat is an external heat source.

13. The method of claim 11 wherein the heat is human body temperature.
